(19) United States
(12) Patent Application Publication
(10) Pub. No.: US 2024/0415797 A1
(43) Pub. Date: Dec. 19, 2024

(54) ENDOPLASMIC RETICULUM STRESS INHIBITOR, NEURODEGENERATIVE DISEASE PREVENTING/IMPROVING AGENT, AGENT FOR PREVENTION/PROGRESSION PREVENTION/IMPROVEMENT OF DEMENTIA, AND FOOD PRODUCT

(71) Applicant: REFINE HOLDINGS CO., LTD., Gifu (JP)

(72) Inventors: Makoto TSUBOI, Tokyo (JP); Yasuko SAKATA, Tokyo (JP)

(73) Assignee: REFINE HOLDINGS CO., LTD., Gifu (JP)

(21) Appl. No.: 18/702,472

(22) PCT Filed: Oct. 19, 2022
(86) PCT No.: PCT/JP2022/038842

§ 371 Date: Apr. 18, 2024

(30) Foreign Application Priority Data
Oct. 20, 2021 (JP) 2021-171332

(51) Int. Cl. A61K 31/23; A61K 36/02; A61P 39/00

(52) U.S. Cl. CPC A61K 31/23; A61K 36/02; A61P 39/00

(57) ABSTRACT

Provided is an endoplasmic reticulum stress inhibitor comprising, as an active ingredient, a triglyceride represented by the following formula (I):

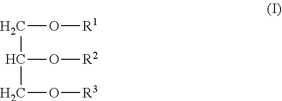

wherein R¹, R² and R³ each denotes a saturated fatty acid residue, at least one of which is a pentadecanoic acid residue. Provided are an endoplasmic reticulum stress inhibitor and a neurodegenerative disease preventing/improving agent, each of which can be taken in the long term and can be used as food, beverage, or a drug for prevention, symptom reduction, and/or improvement.

### CROSS REFERENCE

This application claims priority based on Japanese Patent Application No. 2021-171332 filed in Japan on Oct. 20, 2021, the entire contents of which are incorporated herein by reference in their entirety. In addition, all the contents described in all patents, patent applications, and documents cited in the present application are incorporated herein by reference in their entirety.

### TECHNICAL FIELD

The invention relates to an inhibitor that improves endoplasmic reticulum stress by using a triglyceride composed of saturated fatty acids, mainly pentadecanoic acid. Furthermore, the present invention relates to agents that can prevent, improve, and treat diseases associated with endoplasmic reticulum stress. These agents can be used in various forms, including food products and pharmaceutical compositions.

### BACKGROUND ART

Endoplasmic reticulum stress is a condition that occurs when cells are exposed to various internal or external environmental changes. In this condition, the amount of proteins increases due to abnormal expression of the protein synthesis system in the lumen of the endoplasmic reticulum. This leads to the storage of proteins that cannot be eliminated normally, preventing their normal folding and resulting in their accumulation as bad proteins. Endoplasmic reticulum stress is caused by various factors such as nutrient deprivation, disturbance of intracellular calcium concentration, hypoxia, expression of mutant proteins, and viral infection. During the endoplasmic reticulum stress condition, cells increase lipid synthesis and expand the endoplasmic reticulum to enhance the protein folding capacity and maintain homeostasis. When the accumulation of defective proteins is relatively low, the endoplasmic reticulum stress response which is a response mechanism for eliminating defective proteins, occurs. However, when the stress is severe or persists for a long time, it leads to the accumulation of denatured proteins in the endoplasmic reticulum. This adversely affects the cell. In order to avoid an endoplasmic reticulum stress-induced damage and maintain homeostasis, cell death (apoptosis) is induced in tissues and organs throughout the body. In nervous tissue, this reaction leads to the degeneration and loss of neurons. When this reaction occurs in nervous tissue, degeneration and loss of nerve fibers (neurons) occur. It has been suggested that endoplasmic reticulum stress is involved in developing neurodegenerative diseases (see Non-Patent Literature 1).

With the rise of an aging society, the number of people with neurodegenerative diseases is increasing rapidly, creating a significant social challenge. Neurodegenerative diseases are conditions that damage and cause the loss of certain groups of neurons in the central nervous system, leading to impaired neurological function. Typical neurodegenerative diseases include Alzheimer's disease and Creutzfeldt-Jakob disease, which cause cognitive dysfunction, Parkinson's disease, amyotrophic lateral sclerosis (ALS), and Huntington's disease, which present motor dysfunction. It is known that endoplasmic reticulum stress plays a role in the development of these conditions. The accumulation of degenerated proteins is observed as a common feature of the disease, which is also called folding disease. For example, in Alzheimer's disease, accumulation of amyloid-β extracellularly and tau protein intracellularly is observed. In Parkinson's disease, α-synuclein accumulates; in ALS, mutant superoxide dismutase; and in Huntington's disease, huntingtin protein accumulates. Creutzfeldt-Jakob disease, classified as a prion disease, is caused by the accumulation of abnormal prions. Thus, although the proteins that accumulate differ from disease to disease, there is a commonality in that the accumulation of degenerative proteins causes damage to specific nerve cells and results in cell death.

Various ingredients that are believed to have the potential to prevent dementia through foods have been reported so far, but their efficacy is not yet confirmed. Some foods that are known to inhibit oxidative stress include polyphenols (refer to Non-Patent Literature 2), vitamins C, E, etc. (refer to Non-Patent Literature 3), and folic acid (refer to Non-Patent Literature 4). Antioxidants are believed to prevent neuronal cell death by removing reactive oxygen species generated in the brain as a result of oxidative stress during the pre-dementia stage. It has been reported that the 7-carbon fatty acid and triheptanoin, a triglyceride composed of these fatty acids, can increase circulating ketone bodies and reduce amyloid-3 deposition in patients with Alzheimer's disease (refer to Patent Literature 1). However, the effects of these are unknown and not sufficient.

### PRIOR ART DOCUMENT

### Non-Patent Literature

[Non-patent literature 1] S. Kanemoto and K. Imaizumi, Endoplasmic reticulum stress and disease, Journal of Japanese Biochemical Society 90(1):51-59 (2018).
[Non-patent literature 2] Costa C, et. al. Current evidence on the effect of dietary polyphenols intake on chronic diseases. Food Chem Toxicol 110:286-299 (2017).
[Non-patent literature 3] Bhatti A B, et. al, Vitamin supplementation as an adjuvant treatment for Alzheimer's disease. J Clin Diagn Res, 10, 7-11 (2016).
[Non-patent literature 4] Cacciapuoti F. Lowering homocysteine levels with folic acid and B-vitamins do not reduce early atherosclerosis, but could interfere with cognitive decline and Alzheimer's disease. J Thromb Thrombolysis, 36, 258-262(2013).

### PATENT LITERATURE

[Patent literature 1] JP 2013-516416 A

### SUMMARY OF INVENTION

### Problem to be Solved by the Invention

No known food or drink prevents or improves neurodegeneration by suppressing cell death through endoplasmic reticulum stress reduction and inhibition. In addition, there are problems with drugs for dementia, such as insufficient efficacy and side effects, and there is a need for food, drink, and drugs that can be taken long-term for prevention, symptom reduction, and improvement. The present invention seeks to solve these problems. Therefore, the problem to be solved by the present invention is to provide an endoplasmic reticulum stress inhibitor, a neurodegenerative disease preventing/improving agent, an agent for prevention/progression prevention/improvement of dementia, and a food product that can be used as a food or drug suitable for such applications.

### Means for Solving the Problem

As a result of intensive research and investigation to solve the above problems, we, the applicants found that triglycerides composed of saturated fatty acids mainly containing pentadecanoic acid (C15) (pentadecanoic acid triglycerides: hereinafter referred to as “PdATG”) acts on hippocampal derived neurons to inhibit the accumulation of degenerative proteins in the endoplasmic reticulum caused by stress-induced damage, thereby reducing the resulting cell death (apoptosis) and preventing or improving neurodegenerative diseases, leading to the completion of this invention.

That is, the first aspect of the invention to solve the above problem is an endoplasmic reticulum stress inhibitor comprising, as an active ingredient, a triglyceride represented by the following formula (I):

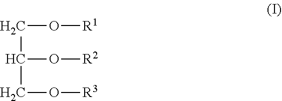

wherein R¹, R² and R³ each denotes a saturated fatty acid residue, at least one of which is a pentadecanoic acid residue.

In one embodiment of the endoplasmic reticulum stress inhibitor according to the present invention, it is preferable that R¹ and R² or R¹ and R³ of the triglyceride represented by the formula (I) are pentadecanoic acid residues. In another embodiment of the endoplasmic reticulum stress inhibitor according to the present invention, any one of R¹, R² and R³ may be a myristic acid residue (C14), a palmitic acid residue (C16) or a margaric acid residue (C17).

In another preferred embodiment of the endoplasmic reticulum stress inhibitor according to the present invention, it may comprise a triglyceride represented by the formula (I) wherein R¹, R² and R³ all denote pentadecanoic acid residues, and a triglyceride represented by the formula (I) wherein any two of R¹, R² and R³ are pentadecanoic acid residues and another is a myristic acid residue or a palmitic acid residue.

Furthermore, in yet another preferred embodiment of the endoplasmic reticulum stress inhibitor according to the present invention, the triglyceride represented by the formula (I) may be derived from algae of the genus Aurantiochytrium or Schizochytrium, and wherein R¹, R² and R³ each denotes a saturated fatty acid residue, at least one of which is a pentadecanoic acid residue. In addition, it may be a mixture containing unsaturated fatty acids derived from algae of the genus Aurantiochytrium or Schizochytrium.

The second aspect of the present invention is a neurodegenerative disease preventing/improving agent comprising the triglyceride represented by the formula (I) as an active ingredient.

The third aspect of the present invention is an agent for prevention/progression prevention/improvement of dementia comprising the triglyceride represented by the formula (I) as an active ingredient.

Furthermore, the fourth aspect of the present invention is a food product comprising the triglyceride represented by the formula (I) as an active ingredient. This food is preferably used, for example, as a health food, functional food, or food for specified health uses, to improve the daily life of people with memory loss.

### Effects of the Invention

The endoplasmic reticulum stress inhibitor according to the present invention can inhibit endoplasmic reticulum stress in mammalian cells, especially neurons, and can be taken on a long-term basis to provide food, drink, and drugs for prevention, symptom reduction, and improvement of the endoplasmic reticulum stress.

### BRIEF DESCRIPTION OF THE DRAWINGS

FIG. 1 shows the inhibitory effect of PdATG on cell death induced by the endoplasmic reticulum stress inducer, tunicamycin. The vertical axis indicates cell viability (%) and the horizontal axis indicates the concentration of tunicamycin treatment. ** and * indicate significant differences compared to control at 1% and 5% significance levels, respectively. + indicates a significant trend.

### EMBODIMENT FOR CARRYING OUT THE INVENTION

Hereinafter, each embodiment of the present invention will be described with reference to the drawings. Each of the embodiments described below is not limiting the invention of the claims, and not all of the various elements and combinations of elements described in each embodiment are essential to the solution means according to the present invention.

### (Active Ingredient)

In this specification, PdATG means an ester of at least one pentadecanoic acid and a glycerol, and includes a triglyceride wherein at least one, preferably any two, of R¹, R² and R³ shown in Formula (I) below, e.g., R¹ and R² or R¹ and R³, and even more preferably R¹, R² and R³, are pentadecanoic acid residues. The binding position(s) of pentadecanoic acid(s) to the glyceride may be any of the positions 1 to 3.

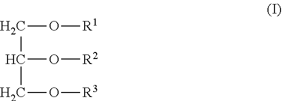

wherein R¹, R² and R³ each denotes a saturated fatty acid residue, at least one of which is a pentadecanoic acid residue.

In the formula, any one of R¹, R² and R³ may be saturated fatty acid residues other than pentadecanoic acid residues. “Saturated fatty acid” is a general term for fatty acids that do not have double or triple bonds within the molecule and is represented by the chemical formula CₙH₂ₙ₊₁COOH. Although not particularly limited, this saturated fatty acid can be straight-chain or branched saturated fatty acids. For example, linear saturated fatty acids, such as capric acid (C10), lauric acid (C12), tridecyl acid (C13), myristic acid (C14), pentadecanoic acid (C15), palmitic acid (C16), margaric acid (C17), stearic acid (C18), arachidic acid (C20) behenic acid (C22), lignoceric acid (C24), and cerotic acid (C26), and branched saturated fatty acids such as 2-hexyl decanoic acid (C16), 13-methylpentadecanoic acid (C16), and 16-methylheptadecanoic acid (C18) are enumerated.

PdATG in one preferred embodiment include both of a triglyceride of formula (I) above, where all of R¹, R² and R³ are pentadecanoic acid residues, and a triglyceride of formula (I) above, where any two of R¹, R² and R³ are pentadecanoic acid residues and the other one is a myristic acid residue or a palmitic acid residue. The content ratio of both in this mixture is not particularly limited, but the mass ratio is preferably 1:2 to 2:1 and more preferably approximately 1:1. In addition, each of these is contained in 10% or more by mass, preferably 20% or more by mass, relative to the total amount of triglycerides. Furthermore, it is more preferred that the mixture of triglycerides containing two or more residues of pentadecanoic acid contains more than 50% by mass of the oil or fat.

In a more preferred embodiment, PdATG is represented by the following general formula (II) or (III).

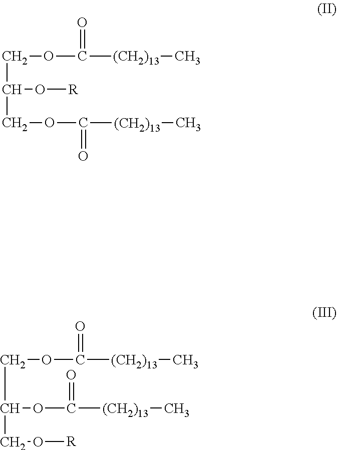

In formulas (II) and (III) above, R is a C₁₄-C₁₆ saturated fatty acid.

It is more preferable that the mixture of triglycerides containing two or more residues of pentadecanoic acid contains 50% by mass or more in the fat and oil, but even if the content of triglycerides containing two or more residues of pentadecanoic acid is 50% by mass or less, the objective can be achieved by increasing the amount of intake. Therefore, the active ingredient of the present invention may exist in the form of a mixture of triglycerides containing two or more residues of pentadecanoic acid, and is at least 1% by mass or more, preferably 50% by mass or more, and more preferably 90% by mass or more in purity relative to the total amount of triglycerides, thereby the mixture itself can exhibit its function as an active ingredient.

The active ingredient of the present invention may exist in the form of a mixture together with triglycerides other than the compound represented by the formula (I), and is at least 1% by mass, preferably 50% by mass or more, more preferably 90% by mass or more, based on the total amount of triglycerides, thereby the mixture itself can exert its function as an active ingredient.

As the active ingredient of the present invention contains at least one, preferably two, or more pentadecanoic acids in its molecule, it is believed that consuming the active ingredient can suppress abnormal proteins that build up in the endoplasmic reticulum due to the endoplasmic reticulum stress. This can reduce cell death and promote normalcy. It is thought that this can help reduce cell death and promote normal cells.

### (Action and Effect)

The active ingredients in this invention act to relieve endoplasmic reticulum stress in various cells, thereby successfully ameliorating diseases in which endoplasmic reticulum stress is the cause of the disease, or ameliorating poor physical condition before leading to a certain endoplasmic reticulum stress-caused disease. In the brains of patients with Alzheimer's disease, deposition of proteins with abnormal structures such as senile plaques and neurofibrillary tangles are observed. The senile plaques are made up of amyloid-3, which is a peptide aggregate of about 40 amino acids that causes neuronal cell death. The deposition of amyloid-3 induces endoplasmic reticulum stress on neurons, which is the main cause of neuronal cell death (See, Ogen-Stern N, et al., Protein aggregation and ER stress, Brain Res, 1648, 658-666(2016)). At the same time, amyloid-R deposition also causes glial cells to produce reactive oxygen species in the brain, which in turn causes oxidative stress that leads to neuronal death. (See, Angelova P R, & Abramov A Y. The interaction of neurons and astrocytes underlies the mechanism of A n-induced neurotoxicity. Biochem Soc Trans, 42, 1286-1290(2014)). It is generally believed that there is a latency period of 20 to 30 years between the deposition of amyloid-3 and the onset of dementia. By the time dementia sets in, a significant number of neurons have already been lost and a substantial percentage of neural circuits have failed. Therefore, it is widely accepted that a complete cure of the disease and regeneration of the brain's neural circuits after the onset of the disease is nearly impossible.

It is crucial to establish preventive measures well in advance of the onset of a disease. One such measure is to take prophylactic and palliative components to prevent neurodegeneration, even before the disease manifests itself. Additionally, early treatment is important from the suspected onset of the disease.

In kidney cells and other cells, B-cells that secrete insulin can experience endoplasmic reticulum stress, leading to impaired insulin production and diabetes mellitus.

### (Age-Related Diseases and Endoplasmic Reticulum Stress)

As described above, endoplasmic reticulum stress progresses due to environmental factors such as aging, genetic factors, and lifestyle, and inadequate repair leads to a gradual loss of homeostasis and cell death. The addition of internal and external factors can lead to age-related diseases, which are characterized by specific diseases in each organ. Among these, metabolic syndrome is a risk factor for cerebrovascular disease, cardiovascular disease, and diabetes mellitus, and increases atherosclerosis, decreased insulin sensitivity, and cancer incidence. Although there is an oxidative stress hypothesis as a risk for aging progression and the relationship between many diseases and reactive oxygen has been debated, the reality is that the endoplasmic reticulum stress is thought to contribute to this risk. The progression of endoplasmic reticulum stress state is a major factor in the development of adult diseases, including age-related diseases and age-related neurodegenerative diseases. This stress state can manifest in different tissues, leading to the onset of cancer and other health problems. Therefore, it has become clear that pharmaceuticals and food ingredients that can improve endoplasmic reticulum stress are essential for maintaining people's health.

The endoplasmic reticulum stress response is one of the mechanisms of cellular stress adaptive response and also plays an important role in cancer cell survival in microenvironments such as hypoxia and low glucose in tumors. Cancer has become the leading cause of death in our country. Cancer is characterized by 1) autonomous proliferative capacity, 2) invasive and metastatic potential, and 3) cachexia (malnutrition due to deprivation of nutrients by the cancerous tissue, no matter how much nutrition is consumed). Cancer cells can survive and proliferate in hypoxic and undernutrition environments. The hypoxic and undernutrition environment induces endoplasmic reticulum stress, causing cancer cells to upregulate molecular chaperones that promote cancer cell proliferation and metastasis. In breast cancer cells and hepatocellular carcinoma, up-regulation of XBP-1 is thought to contribute to the survival of cancer cells. Suppression of XBP-1, which is abnormally expressed due to increased endoplasmic reticulum stress in cells, may also inhibit carcinogenesis. Regarding the XBP-1 (X-box binding protein 1), it is known that whose mRNA is spliced during endoplasmic reticulum stress response, and spliced XBP-1 acts as a nuclear transcription factor to efficiently signal abnormal protein accumulation (Yanagitani, K., et al. Mol Cell vol. 34, 191-200 (2009)).

### (Method for Producing Triglyceride Mixture)

The triglyceride mixture, which is the active ingredient of the present invention, may be chemically synthesized or naturally occurring. If natural, its source is not limited. Sources of naturally occurring triglycerides include lipids produced in the body by living organisms, such as livestock and poultry fats, fish and shellfish oils, vegetable oils, or lipid-producing microorganisms. From the standpoint of industrial productivity, microorganisms such as algae, bacteria, fungi (including yeast), and/or protists are preferred. Preferred microorganisms include those selected from the group consisting of golden algae (such as microorganisms of kingdom Straminipila), green algae, diatoms, dinoflagellates, yeasts, and fungi of the genus Mucor and Mortierella. Members of the kingdom Straminipila include microalgae. Microalgae are photosynthetic organisms that produce oxygen, and their cell size ranges from 1 μm to 100 μm in diameter, excluding mosses, ferns, and seed plants. It also includes Labyrinthula species, which are closely related protists of microalgae. Labyrinthula species are heterotrophic marine eukaryotic microorganisms that do not photosynthesize and are widely distributed mainly in the subtropics and tropics. In general, Labyrinthula species are broadly divided into Labyrinthulidae and Thraustochytriidae, which include the genera Labyrinthula, Aurantiochytrium Aurantiochytrium, Schizochytrium, Thraustochytrium, Aplanochytrium, oblongichytrium, Botryochytrium, Japonochytrium, and others.

The genus Aurantiochytrium, Schizochytrium, or Slaustochytrium is more preferred as the Labyrinthula species to be cultured. These genus have relatively high lipid and other production capacities and can produce hydrocarbons such as squalene, making them suitable for use in food, biofuel feedstock, and other applications.

Cultivation of Labiurintula species may be performed by any culture method, such as batch culture, continuous culture, or flow culture. The culture of Labiurintula species can be performed by any appropriate culture method, such as shaking culture, aeration culture, ventilation agitation culture, airlift culture, and static culture. Among these culture methods, aeration agitation culture or airlift culture is more preferred. For example, mechanical agitation reactors, airlift reactors, filled-bed reactors, fluidized-bed reactors, etc., can be used as culture equipment for culturing Labyrinthula species. Various types of containers such as tanks, jar fermenters, flasks, dishes, culture bags, tubes, test tubes, etc., can be used as culture vessels, depending on the purpose of culture and culture capacity. Culture vessels may be made of inorganic materials such as stainless steel and glass, or organic materials such as polystyrene, polyethylene terephthalate copolymer, polypropylene, or other appropriate materials.

Cultivation of Labiolintula species may be performed under appropriate temperature, pH, and aeration conditions. The incubation temperature may be set to be between 5° C. and 40° C. Between 10° C. and 35° C. is more preferable, and between 10° C. and 30° C. is even more preferable. The pH may be between 2 and 11, preferably between 4 and 9, and more preferably between 6 and 8.

Cultures of Labyrinthula species can be conducted with passages at appropriate intervals, depending on the genus and species of Labyrinthula species, medium composition, and culture conditions. For example, the logarithmic growth phase of Labyrinthula species ends about 2 days after the start of culture, and they enter the death phase in about 7 days. Therefore, it is preferable to perform passages of Labyrinthula species at intervals of 1 to 10 days, more preferable at intervals of 2 to 7 days, and even more preferable at intervals of 2 to 5 days. The incubation time of the Labyrinthula species can be performed as an appropriate time depending on the genus and species of the Labyrinthula species, medium composition, culture conditions, and the purpose of the culture. The genus Aurantiochytrium of Labyrinthula algae is particularly preferred because it is a heterotrophic algae that lives in brackish water and has the characteristic of assimilating nutrients in the water, producing lipids, and accumulating them in the cells.

For algae of the genus Aurantiochytrium, it is preferable to use strains with an excellent ability to produce the desired triglycerides. Such algal strains may be naturally collected and isolated, cloned through mutation induction and screening, or established using genetic recombination technology. Although not particularly limited, for example, Aurantiochytrium Sp. strain SA-96, Aurantiochytrium Sp. strain NIES-3737, Aurantiochytrium strain NB6-3, Aurantiochytrium strain mh0192 and Aurantiochytrium strain mh1959 have the property of accumulating, in their cells, a large amount of triglycerides containing pentadecanoic acid (PDA), which is an odd-chain fatty acid, and those containing docosahexaenoic acid (DHA) and docosapentaenoic acid (DPA), which are highly unsaturated fatty acids.

The cultivation of the above Aurantiochytrium algae is carried out using methods established in the art. That is, ordinary maintenance culture is carried out according to established methods by seeding the algae into a medium with appropriately prepared components. The medium for cultivating Aurantiochytrium essentially contains salts, carbon sources, and nitrogen sources. In general, the so-called GTY medium (10-40 g/L of artificial seawater, 20-100 g/L of D(+)glucose, 10-60 g/L of tryptone, and 5-40 g/L of yeast extract) is used for culturing microalgae.

Carbon sources include sugars such as glucose, fructose, and sucrose. These carbon sources are added, for example, at a concentration of 20-120 g per liter of the medium.

The algae of the genus Aurantiochytrium is a marine algae, and an appropriate amount of artificial seawater is added to the culture medium. Preferably, the artificial seawater is added so that the final salinity of the culture medium is about 10% (v/v) to about 100% (v/v) of seawater (salinity 3.4% (w/v)), e.g., salinity is about 1.0% to 3.0% (w/v).

In general, various nitrogen sources can be added to the culture medium of microalgae, such as organic nitrogen such as monosodium glutamate and urea, or inorganic nitrogen such as ammonium acetate, ammonium sulfate, ammonium chloride, sodium nitrate, ammonium nitrate, etc., or biogenic digests such as yeast extracts, cornstarch liquor, polypeptone, peptone, and tryptone. In particular, cell extracts obtained by extracting liquid components from various animal cells are preferred as nitrogen sources to be added to the medium used for culturing algae of the genus Aurantiochytrium. The use of cell extracts, which are rich in nutrients such as cell-derived amino acids, nucleic acids, vitamins, and minerals, and are available at low cost, is extremely advantageous when cells must be mass cultured on an industrial scale to obtain cultured cell products.

However, when using a medium prepared on the basis of cell extracts as described above, the ratio of odd-chain fatty acids in the triglycerides produced by the cultured algae was significantly reduced, and therefore the cell extracts could not be used as a nitrogen source for the medium when efficiently producing the object of the present invention. Therefore, the present inventors found that the production of odd-chain fatty acids dramatically increased when Aurantiochytrium algae were cultured in an algae culture medium prepared by adding cell extracts treated with strong acid, compared to the case where cell extracts without such treatment were added. The method for producing triglycerides containing odd-chain fatty acids as a major component has already been reported (JP 2017-063633 A).

Furthermore, in one preferred embodiment of the invention, the basic medium for cultivating algae of the genus Aurantiochytrium is a medium with 2% or more glucose, 0.5-4% monosodium glutamate, 0.1-2% yeast extract, 1-3.3% sea salt, and 2-20% whey (animal or vegetable) supplemented with 0 to 50 mM of valine and 0 to 50 mM of sodium propionate. Animal or vegetable whey is preferably tofu whey (soybean whey). To this basic medium, add 2% or more of a culture of Aurantiochytrium that has been pre-cultured for 72 hours at 20-30° C. with 2% or more glucose, 0.5-4% monosodium glutamate, 0.1-2% yeast extract, 1-3.3% sea salt, and 2-20% whey (animal or vegetable). The Aurantiochytrium-added culture medium is aerated and gently agitated, and incubated for 48 to 200 hours at 20 to 30° C. and pH of 5.0 to 8.5 (use 1.0M NaOH solution for pH adjustment). After culturing, Aurantiochytrium cells that produced pentadecanoic acid triglyceride can be recovered by centrifugation (see, the specification of WO 2020/054804).

The pellets collected from the culture medium obtained by the above method by centrifugation or filtration are dried by freeze-drying or drying by heating. Alternatively, the medium in which the algal cells are suspended after cultivation may be used directly for the triglyceride extraction step. Extraction may be carried out multiple times using different organic solvents. As organic solvents, mixtures of polar and weakly polar solvents such as n-hexane/ethanol mixtures, chloroform/methanol mixtures, or ethanol/diethyl ether mixtures can be used. The obtained extracts are purified by methods known to those skilled in the art.

As a method for separating triglycerides, a fractionation method known to those skilled in the art is adopted. Separation and purification may be performed using various physicochemical properties such as the polarity of the triglyceride molecule to be fractionated, the solubility in a solvent, the melting point, the specific gravity, and the molecular weight, and column chromatography technology is preferably used. The conditions for the means of separating the triglycerides can be set by the normal condition study of the person skilled in the art, depending on the composition of the triglyceride mixture and the type of triglyceride to be fractionated.

The algae of the genera Schizochytrium and Aurantiochytrium can synthesize and accumulate both odd-chain fatty acid triglycerides and highly unsaturated fatty acid triglycerides intracellularly. Therefore, ethanol, hexane, or ethyl acetate is added to the obtained algae cells to extract lipids, then the solvent is removed by distillation to obtain the algae lipids. The lipids are allowed to stand at 5° C. to precipitate pentadecanoic acid triglycerides. The composition of the purified pentadecanoic acid triglyceride, “PdATG”, can be analyzed by HPLC-MS, HPLC, gas chromatography, etc.

The algae of the genus Aurantiochytrium can synthesize and accumulate both odd-chain fatty acid triglycerides and highly unsaturated fatty acid triglycerides intracellularly. Therefore, hexane or ethyl acetate is added to the obtained algae cells to extract lipids, then hydrogen peroxide solution is added or ozone is aerated to this lipid solution to oxidatively decompose unsaturated fatty acids. After completion of the reaction, the oxide is removed with sodium hydrogen carbonate and sodium carbonate or an ion exchange resin to obtain pentadecanoic acid triglyceride, “PdATG”. The composition of the purified pentadecanoic acid triglyceride, “PdATG”, can be analyzed by HPLC-MS, HPLC, gas chromatography, etc.

### (Endoplasmic Reticulum Stress Inhibitor)

The endoplasmic reticulum stress inhibitor of the first aspect of the invention includes a formulation that inhibits neuronal cell death due to abnormal protein accumulation in the endoplasmic reticulum. The term “neuronal cell death” used herein refers to both neuronal necrosis and apoptosis. Neuronal cell death here encompasses neuronal necrosis and apoptosis. The term “inhibition of neuronal cell death” encompasses mitigating, reducing, or eliminating neuronal cell death, and inhibiting, preventing or precluding the progression of neuronal cell death. And it can be used to prevent, prevent progression and/or improve the symptoms of disorders (such as diseases and age-related changes) caused by neuronal cell death due to endoplasmic reticulum stress. These disorders may include Alzheimer's disease, Parkinson's disease, amyotrophic lateral sclerosis, Huntington's disease, Creutzfeldt-Jakob disease, amnesia, and so on.

### (Pharmaceuticals)

The neurodegenerative disease preventing/improving agent according to the second aspect of the present invention and the agent for prevention/progression prevention/improvement of dementia according to the third aspect of the present invention contain the pentadecanoic acid triglyceride represented by the above formula (I) as an active ingredient, and can be used to improve symptoms of neurodegenerative diseases such as Alzheimer's disease and the like, and are useful as pharmaceutical products for such purposes. Herein, “pharmaceutical product” means a therapeutic drug for patients with Alzheimer's disease, etc., to prevent, prevent progression of and/or ameliorate symptoms of dementia, etc., by suppressing endoplasmic reticulum stress.

When preparing pharmaceutical products by containing the pentadecanoic acid triglyceride represented by the above formula (I), any pharmaceutically acceptable auxiliary agents, for example, sugars such as dextrin and starch; proteins such as gelatin, soy protein, and corn protein; amino acids such as alanine, glutamine, and isoleucine; polysaccharides such as cellulose and gum arabic; and fats and/or oils such as soybean oil and medium-chain fatty acid triglycerides, etc., may be added to formulate the product into any desired dosage form.

The amount of pentadecanoic acid triglyceride represented by the above formula (I) in the pharmaceutical product is not particularly limited, but it is preferably adjusted so that the intake of pentadecanoic acid triglyceride per adult per day, which is an effective concentration, is about 10 to 1000 mg per day.

The pharmaceutical product of these aspects may contain only the compound of formula (I) as the active ingredient, or may contain other ingredients as long as they do not interfere with the inhibitory effect on neuronal cell death. The other components may be, for example, conventionally used drugs for the treatment or prevention of neurodegenerative diseases. Thus, the endoplasmic reticulum stress inhibitor of the present embodiment provides, in a further aspect, a pharmaceutical composition for preventing and improving neurodegenerative diseases.

### (Foods)

The food product according to the fourth aspect of the present invention contains pentadecanoic acid triglyceride represented by the above formula (I) as an active ingredient, and can be used as a preventive food or drink for a long period before the onset of neurodegenerative diseases, and is useful as a health food for that purpose. Pentadecanoic acid, a component of PdATG, has been reported to be present in small amounts in edible parts such as meat from cattle, pigs, chickens, and sheep, fish from rivers and oceans, and mushrooms, and also in very small amounts in PdATG. Therefore, it is inferred that PdATG is safe based on many years of eating experience.

Therefore, the food of this aspect is useful as a health food taken to promote health. Here, the term “health food” means food or drink that promotes health, prevents the progression of certain health conditions, and alleviates symptoms associated with memory loss, impaired comprehension and judgment, memory impairment, disorientation, executive dysfunction, apraxia of speech, apraxia of action, apraxia of consciousness, and dementia due to aging, and refers to “health food” in a broad sense, including food with functional claims under the “food with health claims system” or food for specified health uses, etc., that meet the standards for safety and efficacy, etc. established by the government.

In the manufacture of the food product containing pentadecanoic acid triglyceride represented by formula (I) above, for example, sugars such as dextrin and starch; proteins such as gelatin, soy protein, and corn protein; amino acids such as alanine, glutamine, and isoleucine; polysaccharides such as cellulose and gum arabic; oils and fats such as soybean oil and medium-chain fatty acid triglycerides can be added to formulate any desired dosage form.

The amount of pentadecanoic acid triglyceride represented by the above formula (I) in the food product of the present invention is not particularly limited, but it is preferably adjusted so that the intake of pentadecanoic acid triglyceride per adult per day is about 1 to 100 mg per day, considering the general intake of the food product to which the triglyceride is added.

Specific examples of the above-mentioned food product include, for instance, beverages such as soft drinks, carbonated drinks, nutritional drinks, fruit drinks, and lactic acid drinks (including concentrates of these beverages and powders for adjustment); ice confections such as ice cream, ice sherbet, and shaved ice; noodles such as soba, udon, harusame, gyoza skin, siumai skin, chinese noodles, and instant noodles; confectionery such as sweet, candy, gum, chocolate, snacks, cookies, jelly, jam, cream, baked goods; processed marine and livestock products such as fish paste, ham, sausage; dairy products such as processed milk, fermented milk; fats and oils and processed foods thereof such as salad oils, tempura oils, margarines, mayonnaises, shortenings, whipped creams, dressings; seasonings such as sauces and bastes; health and nutritional supplements in various forms such as tablets and granules; others such as soups, stews, salads, prepared foods, and pickles.

The food product according to the present invention may contain various food additives, such as antioxidants, flavors, various esters, organic acids, organic acid salts, inorganic acids, inorganic acid salts, inorganic salts, dyes, emulsifiers, preservatives, seasonings, sweeteners, acidifiers, fruit juice extracts, vegetable extracts, nectar extracts, pH adjusters, quality stabilizers, and the like, alone or in combination.

The concentration of pentadecanoic acid triglyceride in the food according to the present embodiment is about 0.00001 to 100% by mass (hereinafter referred to as “%”) as a solid content, and preferably 0.0005 to 50% to obtain usability and good effects.

Examples of the above foods are: for diabetes, foods for people with high blood sugar levels; for eye diseases, foods to improve glaucoma and retinitis pigmentosa; for neurodegenerative diseases, foods to improve dementia, memory, and cerebral blood flow; for Alzheimer's disease, foods to maintain and support memory, which is part of cognitive functions; for Creutzfeldt-Jakob disease, foods for dementia, mental stability, forgetfulness, memory, and cerebral blood flow improvement; for Parkinson's disease, foods for muscle stiffness, reflex disorder, dizziness, dizziness, and insomnia; for Huntington's disease, foods for swallowing difficulty, loss of ability to recognize things (thinking, judgment, and memory), and difficulty in controlling emotions (depression, emotional outbursts, irritability, etc.); for prion diseases, multiple sclerosis, and amyotrophic lateral sclerosis, foods for muscle atrophy, dysarthria, muscle strengthening, and dysphagia; for metabolic diseases, obesity, dyslipidemia/hyperlipidemia, foods for people with high levels of lipids such as cholesterol and neutral fat (triglycerides) in their blood; and for dyslipidemia and hyperlipidemia, where excess lipids in the blood can easily cause arteriosclerosis and increase the risk of myocardial infarction and stroke, are also considered effective in improving these conditions. For hypertension, food for people having various symptoms such as headache, nausea, vomiting, disorientation, convulsions, and high blood pressure; for kidney disorder and chronic kidney disease, food intaking such as when a person is unable to excrete waste through urine and also unable to control the amount of water and salt in the body (body fluid); and food for normalization of growth such as growth of skeletal dysplasia (cartilage formation), but not limited to these.

Next, the present invention will be described in more detail with reference to Examples, but the present invention is not limited to these examples.

### EXAMPLE

### (Production Example 1) Production of Pentadecanoic Acid Triglyceride Using Aurantiochytrium

Aurantiochytrium, strain mh1959 (purchased from Professor Masahiro Hayashi, Faculty of Agriculture, Miyazaki University) was pre-cultured at 25° C. for 72 hours using a medium containing 3.6% glucose, 0.5% monosodium glutamate, 0.2% yeast extract, 1% seawater salt, and 10% whey. This was added to the following basal medium to make it 2% and gently agitated with air. 1 kg of basal medium was prepared by adding 50 mM valine and 25 mM sodium propionate to a medium containing 3.6% glucose, 0.5% monosodium glutamate, 0.2% yeast extract, 1% sea salt, and 10% whey. The culture medium was maintained at 25° C., pH 7.40-7.75 (1.0 M NaOH solution was used for pH adjustment) and incubated for 72-96 hours.

After incubation, the algae were centrifuged at 3000 rpm for 15 minutes and about 20 g of algae were collected. The lipids were extracted by adding hexane or ethyl acetate to 20 g of the obtained algae. Hydrogen peroxide solution was added to the extracted lipid solution (water was added as necessary), and ozone was vented at room temperature. After the reaction was completed, the oxides were removed using sodium bicarbonate, sodium carbonate, or ion exchange resin, and 2 g of pentadecanoic acid triglyceride was obtained, which precipitated as the temperature decreased.

### (Composition Analysis of Pentadecanoic Acid Triglyceride)

Methyl ester of fatty acid (FAME) was obtained by adding 0.50 mL of 14% BF₃-methanol and 0.25 mL of methyl acetate to the lipid containing pentadecanoic acid triglyceride obtained in Production Example 1 and heated at 70° C. for 30 min. Exactly 1.0 mL of n-hexane and 5 mL of saline were added to the reaction solution and mixed vigorously. The mixture was centrifuged at 2800 rpm for 10 min, and the n-hexane layer was used as the sample for gas chromatography.

The above samples were analyzed using a Shimadzu GC-2025 gas chromatograph system. The analysis conditions were as follows: Agilent J&W GC column DB-23 (30 m×0.25 mm) was used, 1 μL sample was injected and detected by FID (hydrogen flame ionization detector) with carrier gas (He, 14 psi). The molecular species of FAME was identified based on the retention time of the fatty acid methyl ester standard product (manufactured by GL Science Co., Ltd.). The fatty acid composition was determined from the area ratio. The obtained component ratios are mass ratios. The ratio of odd-chain fatty acids was determined by multiplying the total amount of fatty acids by the respective ratio (%) of odd-chain fatty acids (C13, C15, C17). The results obtained are shown in Table 1 below.

TABLE 1

| | Retention | | Ratio |
|---|---|---|---|
| Fatty acid | time (min) | Peak area | (mass %) |
| C11:0 | 7.939 | 2207 | 0.3 |
| C12:0 | 8.483 | 2735 | 0.3 |
| C13:0 | 9.065 | 39249 | 5.3 |
| C14:0 | 9.715 | 99919 | 13.4 |
| C15:0 | 10.461 | 422015 | 56.7 |
| C16:0 | 11.291 | 134108 | 18.0 |
| C17:0 | 12.23 | 44681 | 6.0 |

From the results shown in Table 1, the content of odd-chain fatty acids in the triglyceride obtained in Production Example 1 was 68.3% by mass. The fatty acids that make up the triglycerides were found to be mainly pentadecanoic acid residues (C15) and palmitic acid residues (C16).

### (Mass Spectrometry of Pentadecanoic Acid Triglyceride)

Lipids containing pentadecanoic acid triglyceride obtained in Production Example 1 were analyzed by mass spectrometry using a Thermo Fischer Orbitrap mass spectrometer Exactive Plus (AMR DART ion source).

As a result, the fragment composition of the major mass spectral peaks indicated that the pentadecanoic acid triglyceride obtained in Production Example 1 is a triglyceride mixture containing mainly a triglyceride formed only with pentadecanoic acid residues (C15) and another triglyceride containing two units of pentadecanoic acid residues (C15) and one unit of myristic acid residue (C14).

### (Example 1) Examination of the Effect of PdATG on Inhibition of Cell Death by Suppression of Endoplasmic Reticulum Stress Reduction

To examine the effect of PdATG, a solution of pentadecanoic acid triglyceride obtained in Production Example 1 dissolved in ethanol was used as a test drug, and the following experiments were conducted.

### Test Method

Mouse hippocampal neurons (HT22: hippocampal-derived cell line) were seeded in two 10 cm culture dishes at a density of 4×10⁵ cells/dish, and cultured in an incubator at 37° C. and 5% CO₂ for one night. After that, the cells were divided into two groups, one treated with the test drug (50 μg/mL) and the other with solvent (ethanol), and the culture medium was either treated with the test drug or solvent and incubated in a 37° C., 5% CO₂ incubator for 72 hours (3 days). After confirming that both groups were more than 90% confluent (i.e., that the drug treatment had no effect on cell division), the cells were newly seeded into 96-well plates at a density of 4000 cells/well and incubated at 37° C. in a 5% CO₂ incubator.

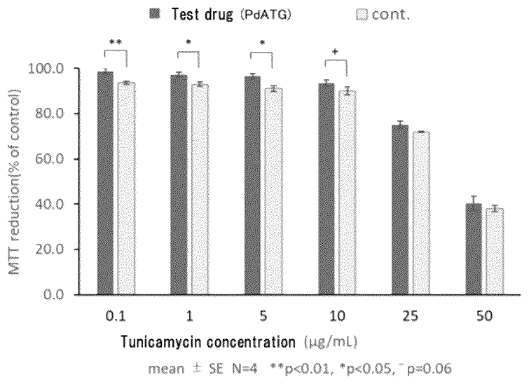

After 24 hours of incubation, tunicamycin (Sigma), an endoplasmic reticulum stress inducer, was added to the medium at concentrations of 0.1, 1, 5, 10, 25, or 50 μg/mL and incubated for 24 hours at 37° C., 5% CO₂ incubator. The survival rate was then measured by the MTT method. The cell viability in the other wells was calculated using the viability of the non-tunicamycin-treated cells in the solvent-treated group as 100%. The results are shown in Table 2 and FIG. 1. Here, the MTT method is a chromogenic detection method where MTT (3-(4,5-dimethyl-2-thiazolyl)-2,5-diphenyl 2H-tetrazolium bromide) is added to the cell culture and the number of viable cells is analyzed by measuring the amount of MTT that is degraded. That is, when cells grown on a culture dish are incubated in MTT solution, water-insoluble formazan dye is produced, mainly depending on mitochondrial enzyme activity. After solubilizing this formazan dye, it is measured in a spectrophotometer. Since the absorbance obtained is proportional to the number of viable cells, the relative degree of cell proliferation can be compared.

TABLE 2

| | Cell viability (%) | | Standard error (SE) | | |
|---|---|---|---|---|---|
| Tunicamycin | Test | Test | Test | Test | |
| concentration | drug | drug | drug | drug | Significant |
| (μg/mL) | (+) | (−) | (+) | (−) | difference |
| 0.1 | 98.8 | 93.7 | 0.92 | 0.98 | ** |
| 1 | 97.3 | 93.0 | 1.38 | 1.27 | * |
| 5 | 96.5 | 91.1 | 1.48 | 1.71 | * |
| 10 | 93.6 | 90.1 | 1.64 | 0.23 | + |
| 25 | 75.1 | 71.9 | 3.22 | 1.50 | |
| 50 | 40.4 | 38.1 | 5.17 | 2.90 | |

**: P < 0.01, *: P < 0.05, +: P = 0.06

The results in Table 2 and FIG. 1 show a concentration-dependent increase in endoplasmic reticulum stress-induced cell death induced by tunicamycin in cells prepared from culture dishes treated with the test drug as well as in cells prepared from solvent-treated culture dishes. However, compared to cells prepared from solvent-treated culture dishes, the increase in cell death (decrease in viability) caused by low concentrations of tunicamycin treatment was significantly suppressed in cells prepared from culture dishes treated with the test drug (when 0.1 μg/mL of tunicamycin was added, significant difference at p<0.01 by Student's t-test), indicating that pretreatment with the test drug confers resistance to endoplasmic reticulum stress-induced cell death.

These results indicate that the present invention can easily prevent and control cell death caused by endoplasmic reticulum stress.

### Example 2

Pancreatic cells in an endoplasmic reticulum stress state can be created by treating pancreatic cells with tunicamycin. When these cells were treated with the pentadecanoic acid triglyceride (PdATG) mixture obtained in Production Example 1, the amount of transcription factor XBP1 gene expressed in said pancreatic cells was measured by real-time PCR. The expression levels are described as relative expression levels when the expression level of normal cells is 100.

TABLE 3

| | | Tunicamycin | Tunicamycin + |
|---|---|---|---|
| | Normal cell | treated cell | PdATG treated cell |
| XBP1 | 100 | 215 | 130 |

The results in Table 2 confirm that the expression level of XBP-1, which was enhanced by the endoplasmic reticulum stress response, approaches the normal state upon the addition of PdATG. This phenomenon may be effective in a variety of cells, and PdATG is expected to ameliorate diseases and cell damage caused by endoplasmic reticulum stress.

### Industrial Utility

The PdATG according to the present invention can be used to prevent, prevent progression of, and ameliorate forgetfulness, impaired comprehension and judgment, memory impairment, disorientation, executive dysfunction, loss of speech, action, or cognition, and dementia, and provides an endoplasmic reticulum stress prevention and suppression agent with no or few, if any, side effects. It is useful as a health food and medicine to prevent the onset of dementia, prevent the progression of symptoms, and alleviate and ameliorate symptoms by preventing and reducing neuronal cell death.

1-9. (canceled)

10. A method of inhibiting endoplasmic reticulum stress, comprising:
administering, to a subject in need thereof, a first triglyceride represented by the following formula (I): 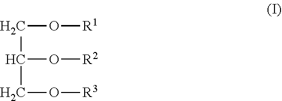
wherein R¹, R² and R³ each denotes a saturated fatty acid residue, at least one of which is a pentadecanoic acid residue.

11. The method according to claim 10, wherein either (i) R¹ and R² are pentadecanoic acid residues, or (ii) R¹ and R³ are pentadecanoic acid residues.

12. The method according to claim 10, wherein any one of R¹, R² and R³ is a myristic acid residue (C14), a palmitic acid residue (C16) or a margaric acid residue (C17).

13. The method according to claim 10,
wherein, in the first triglyceride, R¹, R² and R³ all denote pentadecanoic acid residues,
wherein the method further comprises administering a second triglyceride, which is also represented by the formula (I), and
wherein, in the second triglyceride, any two of R¹, R² and R³ are pentadecanoic acid residues and another is a myristic acid residue or a palmitic acid residue.

14. The method according to claim 10, wherein the first triglyceride is derived from algae of the genus Aurantiochytrium or Schizochytrium.

15. A method of improving or preventing progression of a neurodegenerative disease, comprising:
administering, to a subject in need thereof, a first triglyceride represented by the following formula (I): 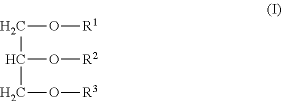
wherein R¹, R² and R³ each denotes a saturated fatty acid residue, at least one of which is a pentadecanoic acid residue.

16. The method according to claim 15, wherein either (i) R¹ and R² are pentadecanoic acid residues, or (ii) R¹ and R³ are pentadecanoic acid residues.

17. The method according to claim 15, wherein any one of R¹, R² and R³ is a myristic acid residue (C14), a palmitic acid residue (C16) or a margaric acid residue (C17).

18. The method according to claim 15,
wherein, in the first triglyceride, R¹, R² and R³ all denote pentadecanoic acid residues,
wherein the method further comprises administering a second triglyceride, which is also represented by the formula (I), and
wherein, in the second triglyceride, any two of R¹, R² and R³ are pentadecanoic acid residues and another is a myristic acid residue or a palmitic acid residue.

19. The method according to claim 15, wherein the first triglyceride is derived from algae of the genus Aurantiochytrium or Schizochytrium.

20. The method according to claim 15, wherein the neurodegenerative disease is dementia.

21. The method according to claim 15, wherein the method ameliorates or prevents progression of at least one symptom selected from the group consisting of forgetfulness, impaired comprehension, impaired judgment, memory impairment, disorientation, executive dysfunction, loss of speech, loss of action, and loss of cognition.

22. A method of preventing a neurodegenerative disease, comprising:
administering, to a subject in need thereof, a first triglyceride represented by the following formula (I): 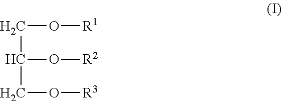
wherein R¹, R² and R³ each denotes a saturated fatty acid residue, at least one of which is a pentadecanoic acid residue.

23. The method according to claim 22, wherein either (i) R¹ and R² are pentadecanoic acid residues, or (ii) R¹ and R³ are pentadecanoic acid residues.

24. The method according to claim 22, wherein any one of R¹, R² and R³ is a myristic acid residue (C14), a palmitic acid residue (C16) or a margaric acid residue (C17).

25. The method according to claim 22,
wherein, in the first triglyceride, R¹, R² and R³ all denote pentadecanoic acid residues,
wherein the method further comprises administering a second triglyceride, which is also represented by the formula (I), and
wherein, in the second triglyceride, any two of R¹, R² and R³ are pentadecanoic acid residues and another is a myristic acid residue or a palmitic acid residue.

26. The method according to claim 22, wherein the first triglyceride is derived from algae of the genus Aurantiochytrium or Schizochytrium.

27. The method according to claim 22, wherein the neurodegenerative disease is dementia.

28. The method according to claim 22, wherein the method prevents at least one symptom selected from the group consisting of forgetfulness, impaired comprehension, impaired judgment, memory impairment, disorientation, executive dysfunction, loss of speech, loss of action, and loss of cognition.
